(19) United States
(12) Patent Application Publication
(10) Pub. No.: US 2011/0223253 A1
(43) Pub. Date: Sep. 15, 2011

(54) PHYSICALLY STABILIZED BIODEGRADABLE OSTEOCHONDRAL IMPLANT AND METHODS FOR ITS MANUFACTURE AND IMPLANTATION

(75) Inventors: Katrin GISSELFÄLT, Olofstorp (SE); Magnus SVENSSON, Bohus (SE)

(73) Assignee: ARTIMPLANT AB, Vastra Frolunda (SE)

(21) Appl. No.: 12/723,740

(22) Filed: Mar. 15, 2010

(51) Int. Cl. A61F 2/28; A61K 9/00; A61K 35/14; A61P 17/04; A61P 19/02; A61P 19/00

(52) U.S. Cl. 424/484; 623/23.61; 424/426

(57) ABSTRACT

A physically stabilized biodegradable osteochondral implant includes a porous matrix element of a resilient material and blood coagulated in vitro in open pores of the element. Also disclosed is a method of manufacture of the implant and a method of restoring a damaged articular surface by use of the implant.

### FIELD OF THE INVENTION

The present invention relates to a biodegradable osteochondral implant, more specifically to a physically stabilized osteochondral implant comprising a biodegradable porous resilient matrix element, and methods for its manufacture and implantation.

### BACKGROUND OF THE INVENTION

Damaged articular surfaces with or without damage in the underlying bone, such as an articular surface of the knee, can be restored by transfer of an osteochondral plug from a neighbouring region that bears no or little weight (for a review, see: Cartilage Restoration, Part 2. Techniques, Outcomes, and Future Directions. J Winslow Alford and B J Cole, Am J Sports Med 33:443-460 (2005). A problem with this method is the limited availability of suitable autografts and also donor site morbidity. Another method for restoration is autologous chondrocyte implantation. In this method normal hyaline cartilage is harvested by biopsy and expanded in vitro. Cartilage remaining at the damaged area is removed so as to leave healthy surrounding hyaline cartilage to form stable vertical walls around a preferably circular cartilage-free area. A periosteal patch of a size fitting into the cartilage-free area of the defect is removed from a suitable non-weight bearing site of the bone and then sewn onto the cartilage so as to cover the damaged articular surface. The expanded chondrocytes are then implanted into the defect by means of a syringe. Another option is an osteochondral allograft transplantation from a suitable donor. More recently various matrix tissue scaffolds have been proposed for the substitution of periostal patches, allowing the in-growth of chondrocytes from neighbouring cartilage and also to transfer cultured cells into the defect.

U.S. Pat. No. 5,876,452 (Athanasiou et al.) discloses a cylindrical biodegradable, porous bioerodible implant device of a synthetic material, such as poly(DL-lactide-co-glycolide), consisting of a bone phase that abuts against the underlying bone for anchoring and a cartilage phase that interfaces with the adjacent layer of articular cartilage. For improved in-growth of cartilage cells the cartilage portion of the matrix contains transforming growth factor-β (TGF-β).

U.S. Patent Appln. No. 2005/0043813 (Kusanagi et al.) discloses an acellular matrix implant for implantation into a cartilage lesion comprising a collageneous, gel-gel, polymer of an aromatic organic acid or a thermo-reversible hydrogel fabricated as a sponge or porous honeycomb scaffold. Also disclosed is a biodegradable sealant of a top or bottom cartilage or bone lesion.

U.S. Patent Appln. No. 2003/0229400 (Masuda et al.) discloses a transplantable osteochondral implant comprising cartilage tissue derived from chondrogenic cells cultured in vitro and having a cell associated matrix, the cartilage tissue being attached to a porous biocompatible support scaffold selected from natural bone, demineralised natural bone, collagen, and bone substitute material.

A polyvinyl alcohol-hydrogel implant for replacing worn-out cartilage surfaces is available on the market under the trade name SaluCartilage™ (SaluMedica, Atlanta, Ga.; www.salumedical.com).

In spite of the various devices and methods for restoration of damaged articular surfaces disclosed in the art there is room for substantial improvement.

### OBJECTS OF THE INVENTION

It is an object of the invention to provide an osteochondral implant in form of a physically stabilized resilient porous biodegradable matrix element intended for restoring a damaged articular surface and, if there be need, also subchondral bone.

It is another object of the invention to provide a method of manufacture of the implant.

A further object of the invention is to provide a method of restoring a damaged articular or other bone surface by means of said osteochondral implant.

Further objects of the invention will become apparent from the following summary of the invention, the description of preferred embodiments illustrated in a drawing, and the appended claims.

### SUMMARY OF THE INVENTION

In this application “biodegredable” comprises all kinds of degradation of an implant or a portion thereof in the living body, such as enzymatic, oxidative, and hydrolytic degradation. Furthermore, in this application, “of the same (polymer) material” relates to the chemical nature of the material but not to its form. “Top”, “bottom”, “lateral” faces or sections etc. relate to their disposition in respect of the bottom of a recess prepared by the surgeon in the bone of a joint for implantation. In this application “physically stabilized” refers to an implant matrix element of a resilient material with open pores, which has been made substantially rigid by filling the pores with blood and allowing the blood to coagulate in the pores.

According to the invention is disclosed an osteochondral implant in form of a physically stabilized porous resilient biodegradable osteochondral implant matrix element. The invention is based on the insight that such stabilization can be achieved by soaking the implant matrix element with blood and making the blood coagulate in vitro in the pores of the matrix. The implant of the invention retains substantially the form and the dimensions of the matrix element from which it is made.

The implant of the invention has preferably cylindrical, conical or other rotationally symmetric form. Other shapes, such as cubes or parallelepipeds tailored specific requirements, are also within the scope of the invention. The implant of the invention may be made in any suitable form and size. Most preferred are cylindrical implants. Cylindrical implants ending in a cone, a frustum of a cone or a hemisphere, possibly with rounded bottom face edges, are also preferred. Their diameter is selected so as to make them fit into recesses, in particular bores, in the bone having a diameter of from 3 or 5 mm to 20 mm or more, in particular of from 8 mm to 12 mm. By cutting (milling) rather than drilling the depression into which the implant is to be inserted into the bone, an implant of any desired form with parallel top and bottom faces can be obtained, substantially in form of a parallelepiped. Such an implant is advantageously cut out from a corresponding sheet material According to the present invention the cutting (milling) of the recess in the bone and of the implant matrix element can be controlled by the same or similar software used in dedicated computer-controlled cutting (milling) apparatus.

The matrix element from which the physically stabilized biodegradable osteochondral implant of the invention is made is porous and of a resilient polymer material. Its porosity is of a kind allowing it to be soaked with blood. The material of the implant thus has a sponge-like nature with open pores. A preferred porosity is one of 50% or more, more preferred of 75% or more, even more preferred of 85% or more, most preferred about 90% or more. The porosity of the implant is such that the pores are open pores or comprise open pores. Open pores are pores in communication with the surface of the implant. It is preferred that 50% or more of the pore volume is comprised by the volume of open pores, more preferred 75% or more, even more preferred 85% or more, most preferred 90% or more. A preferred diameter of an open pore is one that allows blood to pass through it, such as a pore diameter of 4 μm or more, in particular of 10 μm or more. It is preferred that at least 50% of the open pore volume is accessible by pores of a pore diameter of 4 μm or more, in particular of 10 μm or more, more preferred 75% or more, even more preferred 85% or more, most preferred 90% or more.

The biodegradable resilient porous osteochondral implant matrix element of the invention is preferably of a substantially homogeneous polyurethane urea material. It is important to use a polymer material that, while porous, resilient and biodegradable, will preserve its physical structure for extended periods of time so as to provide physical support for in-growing bone and cartilage cells and for expanded chondrocytes with which it may have been seeded, such as for a year and preferably for two years and even three years or more. It is also possible to use in the invention other biocompatible polymer materials with the proviso that they must meet these requirements. Useful materials can be selected, for instance, from the group consisting of (L-lactic acid) and its co-polymers and D-lactic acid and/or glycolic acid (Y S Nam et al., Polymer 20, 1783-1790 (1999); polyglycolide, poly(L-lactic acid), poly(D,L-lactic acid), poly(D,L-lactide-co-glycolide; poly(ε-carprolactone), (DL-lactide-co-caprolactone), poly(glycolide-co-trimethylene carbonate), poly(dioxanone) (S L Ishaug-Riley et al., Biomaterials 20, 2245-2256 (1999); tyrosine-PEG-derived poly(ether carbonate) (C YU et al., Biomaterials 20, 253-264 (1999); poly(ortho esters), copolymers of β-hydroxybutyric acid and hydrovaleric acid, poly(anhydrides), poly(trimethylene carbonate), poly(iminocarbonates) (J Kohn et al., Biomaterials 12, 292-304 (1991); tyrosine-derived polycarbonate (V Tangpasuthadol et al., Biomaterials 21, 2371-2378 (2001); poly(trimethylene carbonate-ε-caprolactone)-block-poly(p-dioxanone) (J-T Hong et al., J Polym Sci: Polym Chem 43(A), 2790-2799 (2005).

The physically stabilized biodegradable osteochondral implant of the invention is intended for implantation into a surgically provided recess in a joint surface extending through the cartilage layer into the subchondral bone. The recess has preferably a substantially flat bottom. The sidewalls of the recess are preferably of a substantially perpendicular extension in respect of the bottom and comprise a lower subchondral bone section and an upper cartilage section. Upon implantation the bottom face of the implant is in abutment with the recess bottom while the side walls of the implant are in abutment with the side walls of the recess. The height of the implant corresponds substantially to the height of the recess. The implant thus has a form so as to substantially fill the recess while not extending from it.

A preferred height of the implant is from 1 mm to 6 mm and even 10 mm, in particular from 2 mm to 4 mm. The width of the implant may vary over a wide range such as from 3 or 5 mm to 20 mm and more, in particular of from 8 mm to 12 mm.

The rigidity of the implant matrix element is substantially increased prior to implantation by soaking the implant matrix element with blood of the patient or animal selected for implantation and coagulating the soaked blood in the pores of the matrix. The so formed rigidified implant is then implanted. By “substantially increased rigidity” is understood a resistance to compression by a factor of 2 or more, in particular of 4 or more. Up to a compression of 25% of a non-stabilized matrix element a load causing such compression, when placed on the physically stabilized implant of the invention, will produce a compression of 50% or less and even of 25% or less of that effected on the non-stabilized matrix element.

According to another important aspect of the invention, the implant matrix element is humidified prior to soaking with blood, such as by contacting it with water or saline or by storing it in a humid atmosphere.

According to a further important aspect of the invention, the humidified implant matrix element is soaked with blood by compressing it, contacting it with blood in a compressed state, and allowing it to expand in contact with blood.

Prior to implantation the blood in the implant is allowed to coagulate. According to a still further important aspect of the invention coagulation is accelerated. Acceleration may be provided by, for instance, supplying energy to the implant, such as in form of radiation, in particular microwave radiation. Acceleration of coagulation may also be provided by contacting the matrix element soaked with blood with a coagulation enhancer, such as human coagulation factor VII.

Thus, according to the invention is provided a method of physically stabilizing a resilient porous biodegradable osteochondral implant matrix element, comprising providing a biocompatible aqueous media; humidifying the implant matrix element by contacting it with the aqueous media; compressing the implant; contacting it with blood obtained from a patient or animal into which the implant is intended to be implanted or blood obtained from a serologically compatible person or animal; allowing the implant to expand in contact with the blood so as to be soaked with it; coagulating the blood in the implant in vitro. In a hydrated porous resilient matrix the coagulation rate of blood drawn into its pores can be controlled by adding a coagulation delaying agent such as heparin to the aqueous humidifying media.

It is preferred for the non-stabilized resilient porous implant matrix element to be contacted with the aqueous media for a given period of time and at a given temperature, such as a time sufficient for obtaining equilibrium hydration at the contact temperature. A preferred temperature is from about 10° C. to about 45° C., more preferred at from about 18° C. to about 40° C. A preferred contact period is from 10 min to 6 h.

According to the invention is also provided a resilient porous biodegradable osteochondral implant physically stabilized by coagulated blood disposed in pores thereof. The physically stabilized implant of the invention is substantially non-resilient. It can be used for implantation into a patient or animal for restoring a damaged articular surface. The three-dimensional shape of the physically stabilized implant corresponds essentially to that of the non-stabilized implant in an uncompressed state.

According to the invention is furthermore provided a method of restoring a damaged articular surface. The method comprises providing a recess in the surface extending over substantially the entire area of damage and extending into the subchondral bone over substantially the same area; providing a physically stabilized implant of the invention of form mirroring the form of the recess; optionally securing the implant to the adjacent bone and/or cartilage. Securing may be obtained, for instance, by any of suture, staple, pin, adhesive, such as fibrin glue, hook means, and combinations thereof.

In essentially the same manner as for restoration of cartilage and bone of a load bearing site in a joint the present invention can also be applied to donor site augmentation, that is, to restore cartilage and bone of a non-load bearing site, such as one from which a osteochondral plug has been removed.

The invention will now be explained in greater detail by reference to a number of preferred embodiments illustrated in a rough drawing.

### SHORT DESCRIPTION OF THE FIGURES

FIG. 1 is a perspective view of a prior art cylindrical implant matrix element;

FIG. 1a is a corresponding axial sectional view;

FIG. 1b is an enlarged portion of FIG. 1a showing open pores;

FIGS. 2a-2e illustrate the hydration of a cylindrical implant matrix element of the invention;

FIGS. 3a-3e illustrate the loading of a hydrated cylindrical implant matrix element with blood and the formation of the physically stabilized implant of the invention by coagulation of the blood-loaded matrix element;

FIGS. 4-6 are sectional views illustrating the implantation of the physically stabilized implant of the invention in a joint with a damaged articular surface;

FIGS. 7a and 7b illustrate the effect of hydration of a cylindrical implant matrix element on the penetration of blood into the matrix by soaking (FIG. 7a, hydrated matrix; FIG. 7b, dry matrix).

### DESCRIPTION OF PREFERRED EMBODIMENTS

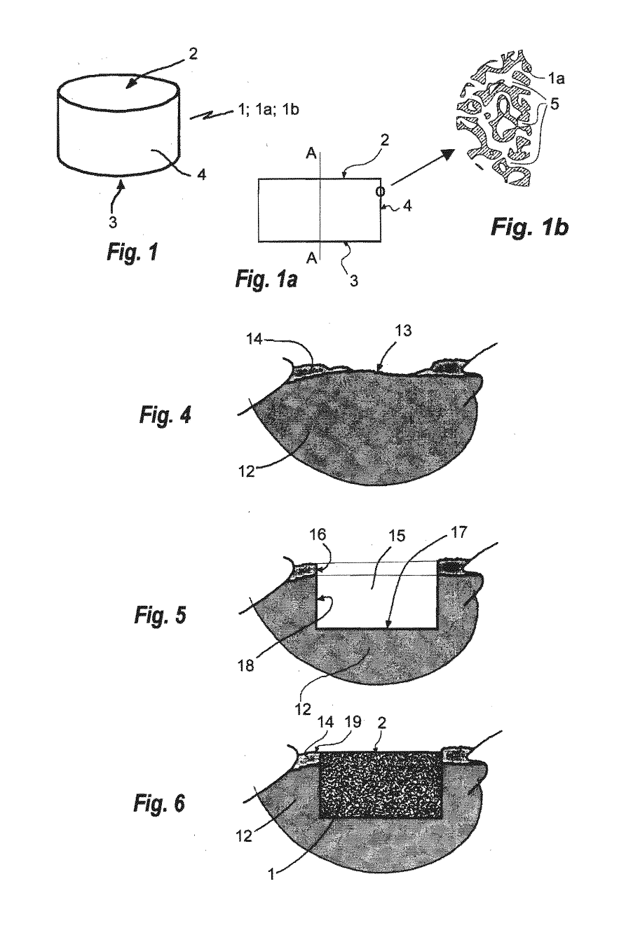

A resilient porous biodegradable cylindrical osteochondral implant matrix element 1a illustrated in FIGS. 1, 1a comprises a flat top face 2, a flat bottom face 3, and a cylindrical lateral face 4 equidistant from rotational axis A-A. The implant matrix element 1a is of a polyurethane urea material (Artelon®, Artimplant AB, Vastra Frolunda, Sweden) with pores 5 opening at the faces 2, 3, 4 (enlarged partial view, FIG. 1b). Prior to implantation the implant matrix element 1a is hydrated by soaking it with water or saline. For soaking the implant matrix element 1a is compressed to remove air from the open pores 5, then immersed into the soaking media, and allowed to there to expand. After reaching a state of hydration equilibrium, excess water or saline, respectively, is removed by compressing the implant matrix element 1a so as to form a hydrated resilient biodegradable cylindrical osteochondral implant matrix element 1b.

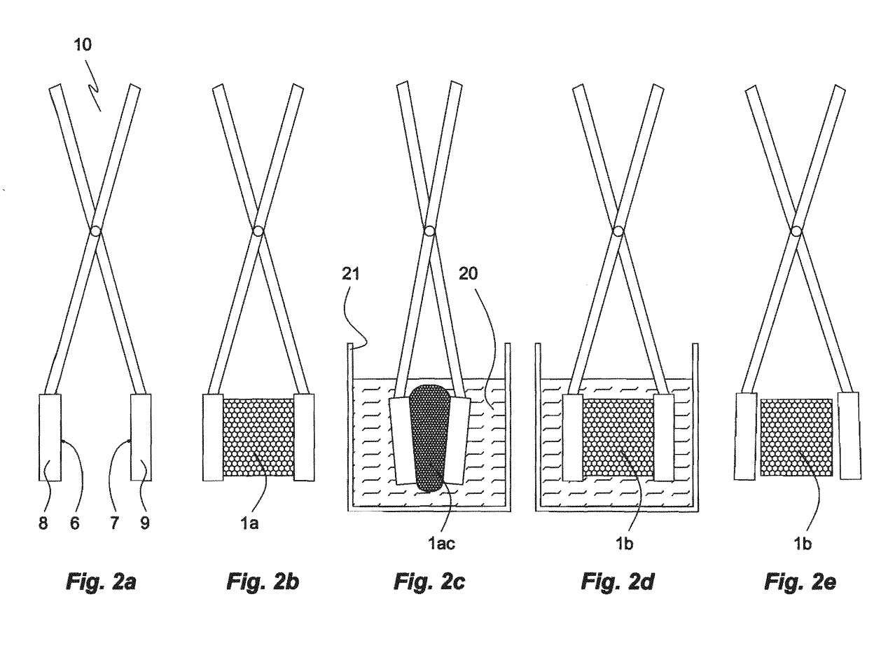

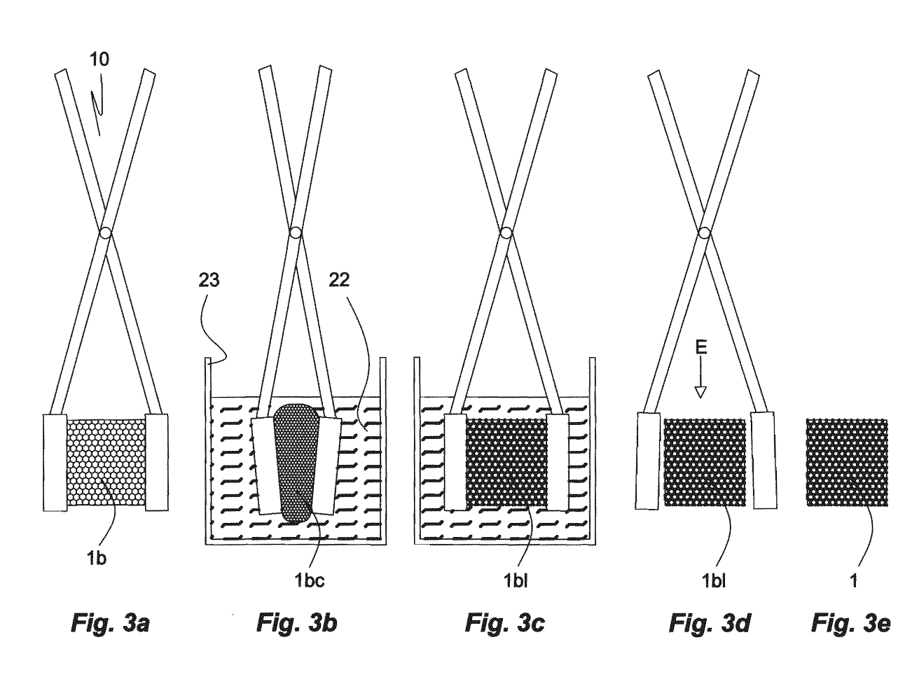

The manufacture of a stabilized resilient biodegradable porous cylindrical osteochondral implant of the invention 1 from the matrix element 1a is illustrated in FIGS. 2 and 3.

In an exemplary manner, the compressed state of the resilient biodegradable cylindrical osteochondral implant matrix element 1a (“matrix element”) is obtained by disposing the matrix element 1a between upper 6 and lower 7 faces of upper 8 and lower 9 brackets, respectively, of a matrix compression tool, such as pincers 10 (FIGS. 2a, 2b). The matrix element 1a loosely held by the pincers 10 is immersed into saline 20 disposed in an open container 21. The brackets 6, 7 are then displaced towards each other until firm resistance by the compressed implant matrix element 1ac is met (FIG. 2c). The compressed implant matrix element 1ac is allowed to expand in the saline so as to soak saline 20 into its expanding pores 5 (FIG. 2d). The saline soaked implant matrix element 1a is kept for one hour in the saline 20 so as to form a hydrated implant matrix element 1b. The hydrated implant matrix element 1b is removed from the container 21. Excess saline 20 is expelled from the pores 5 of the hydrated matrix element 1b by compressing it by means of the pincers 10 (not shown). The hydrated implant matrix element 1b devoid of excess saline is allowed to expand by loosening the grip on the pincers 10 (FIG. 2e). It can be kept for a desired period of time in a hydrated state in a humid atmosphere or be used directly.

The hydrated implant matrix element 1b devoid of soaking media 20 is soaked with blood 22 in an open container 23 in essentially the same manner as when soaking the dry implant matrix element 1a with saline 10 (FIGS. 3a-3e). Blood 22 used for soaking is freshly drawn from the person or animal selected for joint cartilage repair. The compressed hydrated implant matrix element 1bc is allowed to expend immersed in blood. Upon full expansion a hydrated implant matrix element 1bl soaked with blood 22 is obtained (FIG. 3c), which is removed from the container 23 and may be rinsed shortly with water or saline to remove blood from its surface. Prior to implantation blood 22 in the pores 5 of the loaded implant matrix element lbl is allowed to coagulate to form the physically stabilized resilient porous biodegradable osteochondral implant 1 of the invention (FIG. 3e). Coagulation can be accelerated by, for instance, adducing energy to the implant matrix element lbl loaded with blood, such as by irradiation with IR or microwave radiation (FIG. 3d).

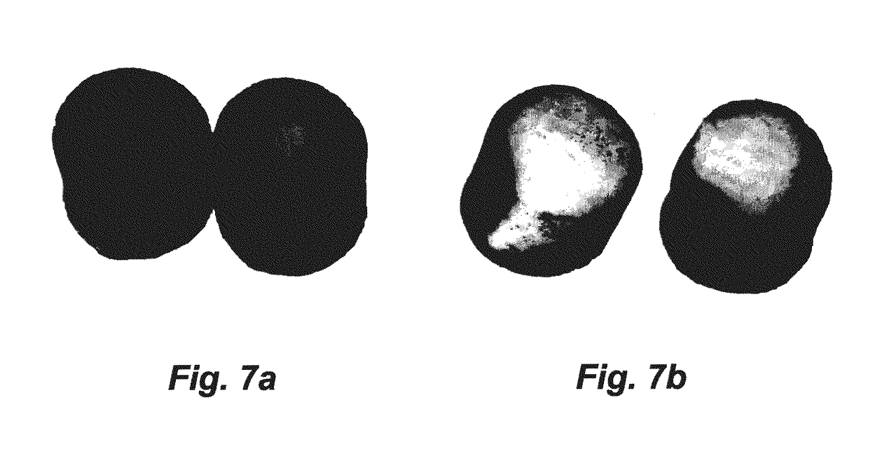

In the non-hydrated (dry) state of the implant matrix element 1a blood soaked into it will start coagulating immediately upon contacting a dry implant matrix element surface. Thereby outer pores, that is, pore sections disposed near the faces 2, 3, 4, will be occluded and prevent blood cells, in particular erythrocytes, from passing into inner pores, that is, pore sections disposed at a distance from the faces 2, 3, 4. The result of soaking non-hydrated implants matrix element 1a specimens and hydrated implant matrix element 1b specimens with blood is shown in FIGS. 7a and 7b, respectively. The white inner zones of the soaked dry implant matrices of FIG. 7a represent inner pore sections not filled with blood due to rapid coagulation of blood in outer pore sections preventing blood cells from penetrating deeper into the matrix.

The implantation of a physically stabilized resilient porous biodegradable osteochondral implant 1 of the invention into an articular bone is illustrated in FIGS. 4-6.

FIG. 4 illustrates a load-bearing portion of a bone 12 pertaining to a joint comprising a damaged articular area 13 substantially free from hyaline cartilage 14. The invention aims at restoring the cartilage in this and similar areas of defective bone surface. For this reason a cylindrical bore 15 is cut into the bone 12 (FIG. 5) covering the entire damaged area 13 and radially extending into the surrounding cartilage 14 to form a circumferential cartilage wall section 16 extending, at the one hand, from the subchondral bone 12/cartilage 14 border in an axial direction towards the joint and, at the other hand from said border in the opposite direction into the bone 12. The diameter of implant 1 is selected so as to correspond to that of the bore 15. Next the implant 1 is inserted into the bore 15 with its free end face 3 ahead until the face 3 is abutting the bottom 17 of the bore 15 (FIG. 6). The implant 1 is so dimensioned that, in an implanted state, its top face 5 is substantially flush with the joint surface 19 of the cartilage 14 surrounding the implant 1. Slow biodegradation of the polyurethane urea scaffold over time allows it to be fully replaced by healthy cartilage and osseous tissue so that the damaged joint area is fully restored.

1. Physically stabilized biodegradable osteochondral implant comprising a porous matrix element of a resilient material and blood coagulated in vitro in open pores thereof.

2. The implant of claim 1 of parallelepipedal form.

3. The implant of claim 1 or cylindrical form.

4. The implant of claim 1 of a porosity of 50% or more.

5. The implant of claim 1 of a porosity of 75% or more.

6. The implant of claim 1, wherein at least 50% of the pore volume is in communication with the environment via pores of a diameter of 4 μm or more.

7. The implant of claim 1, wherein at least 50% of the pore volume is in communication with the environment via pores of a diameter of more than 10 μm or more.

8. The implant of claim 1 of a polyurethane urea material.

9. A method of forming a non-resilient physically stabilized osteochondral implant from a resilient porous biodegradable matrix element, comprising: providing said matrix element cut or otherwise formed to size; compressing the matrix element so as reduce its open pore volume by 50% or more; contacting the compressed matrix element with human or animal blood; allowing the compressed matrix element to expand in contact with the blood so as to be soaked with it; coagulating the blood in the matrix element in vitro to form the physically stabilized osteochondral implant.

10. The method of claim 9, wherein the coagulation rate of the blood in the matrix element is increased by adducing energy.

11. The method of claim 9, wherein the matrix element is hydrated prior to compression.

12. The method of claim 11, wherein hydration comprises compressing the matrix element so as reduce its open pore volume by 50% or more; contacting the compressed matrix element with an aqueous humidifying media such as water or saline; allowing the matrix element to expand in contact with the humidifying media so as to be soaked with the media; optionally storing the soaked matrix element for a desired period of time; compressing the soaked matrix element to remove excess humidifying media.

13. The method of claim 12, wherein the humidifying media comprises a coagulation delaying agent such as heparin.

14. The method of claim 9, wherein the blood is obtained from a patient or animal into which the implant is intended to be implanted or from a serologically compatible person or animal.

15. A method of restoring a damaged articular surface in a person or animal, comprising providing a recess in the bone with the damaged articular surface of an extension at least corresponding to the extension of the damage; providing a physically stabilized biodegradable osteochondral implant of claim 1 corresponding in form to that of the recess; disposing the implant in the recess; optionally securing the implant disposed in the recess to adjacent cartilage and/or bone.
